(19) United States
(12) Patent Application Publication
(10) Pub. No.: US 2023/0277718 A1
(43) Pub. Date: Sep. 7, 2023

(54) WOUND TREATMENT DRESSING, METHOD OF OBTAINING AND USES THEREOF

(71) Applicant: FALCON EMPIRE LDA., Castelo Branco (PT)

(72) Inventors: Filipe José NUNES AGOSTINHO, Castelo Branco (PT); Paulo Jorge GARCIA FRADE, Santa Iria De Azoia (PT)

(21) Appl. No.: 17/999,278

(22) PCT Filed: May 18, 2021
(86) PCT No.: PCT/IB2021/054271

§ 371 Date: Nov. 18, 2022

(30) Foreign Application Priority Data
May 18, 2020 (PT) 116388

(51) Int. Cl. A61L 15/46; A61L 15/60; A61L 15/28

(52) U.S. Cl. CPC A61L 15/46; A61L 15/60; A61L 15/28; A61L 2300/40

(57) ABSTRACT

The present disclosure relates to a wound treatment dressing in which an absorbent layer comprises an odour-inhibiting mixture; wherein the odour-inhibiting mixture comprises coffee grounds. More particularly, a wound treatment dressing is provided for the absorption of odours in wounds, including pressure ulcers, leg ulcers, cancerous wounds, diabetic foot ulcers, burns, traumatic or surgical wounds, which comprises an odour-inhibiting mixture with coffee grounds.

### TECHNICAL FIELD

The present disclosure concerns a wound treatment dressing, in particular to prevent/eliminate odours, which comprises a textile material with the incorporation of coffee grounds to neutralize odours from exudates from chronic wounds. The selection of coffee grounds as a functional agent to be incorporated into the textile was related to its odour neutralizing properties, as well as its potential valorisation as a waste.

Namely, a wound treatment dressing, in particular for the absorption of odours in wounds, including pressure ulcers, leg ulcers, cancerous wounds, diabetic foot ulcers, burns, traumatic or surgical wounds, which comprises an odour-inhibiting mixture with coffee grounds.

### BACKGROUND

Acute and chronic wounds affect about 2% of the population, representing close to 4% of total health care expenditure and approximately 68% of nurses' working time. Of all the symptoms of the wound, the odour is referred by patients and caregivers as the most pungent, causing social isolation, depression, and repulsion.

The bad odour arises from a combination of factors, including bacteria, necrotic tissue, high levels of exudate and poorly vascularized tissue. It is caused by a mixture of volatile compounds, which includes short-chain organic acids (n-butyric, n-valeric, n-caproic, n-heptanoic and n-caprylic), produced by anaerobic bacteria, together with a mixture of amines and diamines, such as cadaverine and putrescine, which are produced in the metabolic processes of other proteolytic bacteria. This odour has been linked to the smell of rotting meat. Recently, dimethyltrisulfide has also been identified in chronic wounds as a source of odour. This compound was found in volatiles released from certain vegetables, fermented milk and aged foods or drinks, being produced by aerobic bacteria such as Pseudomonas aeruginosa.

The smell is difficult to identify, as any odour can be made up of hundreds of different chemicals. It was believed that humans could adapt to any odour, however in the presence of putrescine and cadaverine they tend to feel like coughing, due to the high toxicity of these compounds.

Recently, Gethin et al. evaluated the performance and effectiveness of some topical wound agents, as well as some odour control compounds, present in the environment to which patients are exposed. With this study, they found that activated carbon dressings are the most used, as they are more effective in eliminating bad odour from the wound (about 48.8%), while coffee and coal are two of the products referred by users as potential odour neutralizers.

Coffee is one of the most popular and appreciated drinks worldwide, being consumed for its stimulating and refreshing properties, which are defined by the composition of the coffee tree and the changes that occur during the roasting process. As a result of this large market, the coffee sector is responsible for producing large quantities of waste—spent coffee grounds (SCG) and coffee silverskin (CS). SCGs correspond to the powder that remains after the extraction of the drink—grounds, being a resource found globally, which is given little importance.

SCG represent the most abundant and non-edible agricultural waste obtained by the wet processing of red berries of coffee. Caffeine and chlorogenic acid derivatives (CGA) are found not only in coffee but also in wasted residues, even presenting therapeutic properties. CGA, especially, are of great importance to humans due to their antioxidant properties [L. F. B. Giraldo, “Extraction and characterization of polysaccharides and phenolic compounds from spent coffee grounds and their incorporation into edible films/coatings for food applications”, 2016].

Coffee residues, in addition to those mentioned, contain large amounts of organic compounds (fatty acids, lignin, cellulose, hemicellulose and other polysaccharides) that justify the valorisation thereof. Some researchers have already analysed coffee waste as a biological resource for various purposes, such as for the production of biodiesel, as a source of sugars, as a precursor to the production of activated carbon, as a compound and as an adsorbent for the removal of metal ions.

The removal of pollutants from heavy metals, such as lead, for example, has been of great research interest due to the significant risks of the pollutants to human health and the environment. Of the various existing techniques, adsorption was considered the most practical, inexpensive, and efficient method for reducing lead and other heavy metal pollutants. Ayucitra et al. evaluated and used various residues as precursors of bioadsorbents in an attempt to eliminate contaminants from wastewater. Roasted coffee was analysed as a potential alternative material for bioadsorption due to its high carbon content (around 50.6%).

Gizaw et al. found that SCG are made up of a complex community of non-Saccharomyces yeasts. As such, they are a good substrate to house other yeast species, which indicates that they provide a favorable environment for their development and proliferation. In this sense, it is not advantageous that SCGs are directly in contact with the exudate of chronic wounds, as they can cause an aggravation of the wound. It is suggested the insertion of an antimicrobial agent or the sterilization of SCG to eliminate this problem.

It is necessary to continue exploring the application of SCG and CS, taking into account their profitable use, adding the value of these unused materials and reducing their impact on the environment. Although some characteristics of these compounds have already been reported recently in the literature, there is no study that presents a complete characterization of the two materials.

Wound care has evolved since ancient times. Initially, the application of curative materials aimed at inhibiting bleeding and protecting the wound from environmental irritation, as well as water and electrolyte disturbances. The skin plays an important role in homeostasis and in preventing invasion by microorganisms and as such it needs to be covered much of the time with a bandage after the injury. There are three categories of dressings: biological, synthetic, and biological-synthetic. In the case of biologicals, some of them have disadvantages, such as high antigenicity, low adhesion and risk of cross contamination. Synthetic dressings have a long lifespan, induce a minimal inflammatory reaction and have almost no risk of pathogen transmission. Bio-synthetic dressings have bilayers and are made of high density polymers and biological materials.

These three categories of dressings are all used frequently in clinical settings, but all of them have disadvantages.

An ideal dressing should maintain a moist environment at the wound interface and allow exchange of gases, acting as a barrier to microorganisms and removing the excess exudates. It should also not be toxic, allergenic, sticky and should be easily removed without trauma. In addition, it should be made based on a biomaterial easily accessible, requiring minimal processing, having antimicrobial properties.

Various products such as gauze, hydrogels, foams, hydrocolloids (carboxymethylcellulose), alginate, chitosan, collagen, cellulose, cotton/rayon, transparent films (polyurethane) are recommended as passive dressings for wounds and burns, due to their actions in the local cell responses.

In recent years, a large number of research groups have devoted themselves to producing new and improved wound dressings, synthesizing, and modifying biocompatible materials. Current strategies are also focused on accelerating wound repair by systematically engineered healing materials. In particular, efforts are focused on the use of biologically derived materials, such as chitin and its derivatives, which are able to accelerate healing processes at molecular, cellular and systemic levels.

Chitin is an easily available and inexpensive biological material, obtained from the skeleton of invertebrates, as well as from the cell wall of fungi. It is a copolymer of units of N-acetyl-D-glucosamine linked by glycoside linkage (1-4), wherein the units of N-acetylglucosamine are predominant in the polymer chain. Chitin and its derivatives, such as chitosan, are biocompatible, biodegradable, non-toxic, antimicrobial and moisturizing. Due to these properties, they have good biocompatibility and positive effects on wound healing. However, the low solubility of chitin causes some limitations in its applications.

These biopolymers have been studied in several applications as a raw material not only for absorbable sutures, wound dressings and synthetic fibers, but also in the environmental area in the treatment of effluents. Previous studies show that chitin-based dressings can accelerate the repair of different tissues, facilitating the contraction of wounds and regulating the secretion of inflammatory mediators. Chitosan demonstrated a good effect on the wound healing/cicatrisation process. Chitin can also be used as a coating on normal biomedical materials [S. Pokhrel, P. N. Yadav, and R. Adhikari, “Applications of chitin and chitosan in industry and medical science: A review”, Nepal J. Sci. Technol., Vol. 16, no. 1, pp. 99-104, 2015].

Another problem, found in current commercial dressings, is the fact that it is not as effective in the most peripheral areas. In this sense, the treatment of wounds using ground coffee has been recognized as an alternative medicine, considering its effectiveness in wound healing. Yuwono (2014), after conducting a study in this area, stated that the use of coffee powder as a wound dressing can have a strong influence on the emergence of a new paradigm for the treatment of wounds. This is because the coffee powder promotes a more prolonged action since it easily absorbs the fluid from the wound and can confer antioxidant activity and antibacterial properties to it. Thus, wound healing is more rapid and prevents the risk of contamination and microbial infection. In addition, it allows the absorption and neutralization of odour, minimizes pain, is more cost-effective and does not cause adverse reactions. In terms of the deodorant action, coffee as it is known has a characteristic fragrance, so it is very useful to mask the odour of wounds. These results provide confidence for scientific research in the use of coffee grounds, safely, in a wound treatment dressing [H. S. Yuwono, “The New Paradigm of Wound Management Using Coffee Powder”, Glob. j. Surg., Vol. 2, no. 2, pp. 25-29, 2014].

Many health professionals from areas of coffee plantations have also reported that coffee powder on acute or chronic wounds (diabetes mellitus, sharp cuts, burns) does not cause infections. That is, the coffee powder can come in contact with the wound until its healing. Perry et al. tested the effect of coffee grounds from East and Southeast Asia and state that they can be used to treat burns.

Document BR10201202916 discloses a composition and a formulation thereof for topical use of coffee oil base with high capacity for wound healing. The composition presented is able to keep the scar area with ideal humidity and dry and protected outlying areas, in addition to presenting a systemic action, factors that accelerate the healing process.

In 2014, Hung et al. developed fibers with coffee residues, comprising a polymeric granulate, in order to combat unpleasant human odours released by different sources (biological decomposition, chemical agents, tobacco smoke, etc.) to the environment. The production of this yarn included the compression of a material based on carbonized particles from ground coffee beans and coffee grounds, without organic content. The types of polymers used varied between polyester, nylon or polyethylene terephthalate in fabric, non-woven fabric or knitwear. This invention is intellectual property of the S.Café® brand, its application being essentially used in sports clothing with the intrinsic properties of quick drying, sweat odour control and moisture reduction.

In turn, also in 2014, Carlucci et al. created absorbent personal hygiene articles to reduce related bad odours, acting directly on the released compounds or indirectly on the users' olfactory system. As hygiene items they included sanitary towels, daily pads, adult incontinence articles, baby diapers, paper towels, bath tissues and facial tissues that are used to absorb and retain body fluids and other exudates excreted by the human body. The term “absorbent article” has been used in a very broad sense including any article capable of receiving and/or absorbing and/or containing and/or retaining fluids and/or exudates, especially body fluids/body exudates. In addition, these articles were planned to be disposable, that is, for single use and in order to be compatible with the environment. There are numerous substances that have the capacity to absorb/adsorb and neutralize unpleasant odours, including zeolites, starch, activated carbon, cyclodextrins, chitin or chitosan. In addition, there are functional groups such as esters and aldehydes with this functionality, wherein in this case the aldehydes proved to be more useful ones.

The Carboflex® commercial dressing, marketed by Convatec®, is a sterile, non-adhesive dressing designed for the management of malodorous wounds. It has five layers, one of which consisting of activated carbon, with a high odour retention surface.

The Actisorb Silver 220° commercial dressing, marketed by Systagenix®, consists of an activated carbon knitted fabric, impregnated with silver, which in turn is wrapped in a non-woven nylon.

These facts are described in order to illustrate the technical problem solved by the embodiments of this document.

### GENERAL DESCRIPTION

The present disclosure relates to a wound treatment dressing, with incorporation of coffee grounds for odour neutralization, preferably derived from chronic wound exudates although capable of being used in any malodorous wound. The selection of coffee grounds, as a functional agent to be incorporated into the textile, was related to its odour masking properties, as well as its potential valorisation as a waste.

Therefore, one of the main objectives of this disclosure was to assess the potential for incorporating coffee grounds into a textile material (woven or non-woven fabric) as a functional agent for neutralizing odours from wound exudates.

In an initial phase, the selected material was characterized—coffee grounds, for incorporation into the textile material. After the characterization of the functional agent, it was incorporated into several textile substrates and their respective characterization in terms of odour adsorption/neutralization capacity, using gas chromatography with flame ionization detection (GC-FID). At the same time, a sensory evaluation panel was used to assess the intensity of the odour released by the developed dressings. In addition to these tests, the rate of dehydration and the absorption capacity of the dressings were determined, using an ionic solution that simulates wound exudate. Regarding the skin contact that this disclosure may have, the loss of transepidermal water and skin erythema was evaluated through corneometry tests.

Throughout the development of this disclosure, the most common commercial solutions for this purpose, based on activated carbon, were tested, always establishing a comparison with the results obtained with this disclosure.

A wound treatment dressing is described which comprises: an absorbent layer comprising an odour-inhibiting mixture; wherein the odour-inhibiting mixture comprises coffee grounds.

In a preferred embodiment, the granulometry of ground coffee grounds varies between 3-5000 μm; preferably between 4-4000 μm; more preferably 5-3000 μm and the coffee grounds are dried and/or ground.

The measurement of granulometry/particle size/coffee grounds granule can be performed in several ways, in this disclosure the measurement of granulometry/particle size was carried out through the Leica DM 2500M optical microscope, using the Leica Application Suite software.

In an embodiment, the odour-inhibiting mixture comprises 10-40% (w/V_{inhibiting mixture}) of coffee grounds; preferably 15-30% (w/V_{inhibiting mixture}); more preferably 20-25% (W/V_{inhibiting mixture}).

In an embodiment, the absorbent layer comprises between 0.05 to 5 g of coffee grounds per cm² of absorbent layer; preferably between 0.02 to 2.5 g of coffee grounds per cm² of absorbent layer.

In a preferred embodiment, the dressing further comprises: an outer layer comprising a textile substrate, which serves as a support for the absorbent layer; an absorbent layer comprising a textile substrate, which serves as a support for the inhibiting mixture.

In an embodiment, the odour-inhibiting mixture comprises an adhesion agent in which the odour-inhibiting mixture comprises 0.5-3% (V/V_{inhibiting mixture}) of the adhesion agent; and 20-25% (W/V_{inhibiting mixture}) of coffee grounds.

In an embodiment, the odour-inhibiting mixture comprises an adhesion agent in which the odour-inhibiting mixture comprises 0.5-5% (V/V_{inhibiting mixture}) of the adhesion agent; and 20-25% (W/V_{inhibiting mixture}) of coffee grounds.

In an embodiment, the odour-inhibiting mixture further comprises a dispersant, a binding agent, and a buffer solution.

In an embodiment, the absorbent layer further includes an active ingredient, a moisturizer, a disinfectant, an anesthetic agent or combinations thereof.

In an embodiment, the absorbent layer comprises between 0.05 to 5 g of coffee grounds per cm² of absorbent layer.

In an embodiment, the absorbent layer is coated using a Meyer bar and/or film applicator wherein the wire diameter used in Meyer bar rod comprises between 100-110 μM.

In an embodiment, the dressing further comprises an outer layer that comprises a textile substrate, which serves as a support for the absorbent layer carrying the inhibiting mixture.

In an embodiment, the absorbent layer further comprises superabsorbent polymers.

In an embodiment, the adhesion agent is selected from a list comprised of: a highly active matrix based on anionic, cationic, nonionic, amphoteric agents or combinations thereof, preferably based on siloxanes.

In an embodiment, the binding agent is selected from a list comprised of: chitosan, gelatin, pectin, cellulose, cellulose derivatives, glucomannan, acrylic emulsions, styrene-butadiene emulsions, styrene-acrylic emulsions, polyurethane-based dispersions, or combinations thereof.

In an embodiment, the dispersant is selected from a list comprised of: polyester, siloxane, polyphosphates, polyacrylates, or combinations thereof, preferably a copolymer of polyester and siloxane.

In an embodiment, the textile substrate is a natural fiber, a synthetic fiber or combinations thereof.

In an embodiment, the pH range of the buffer solution is comprised between 6 and 10.

In an embodiment, the textile substrate comprises cotton (CO), polyester (PES), non-woven fabric (TNT), or combinations thereof.

In an embodiment, the textile substrate comprises 100% CO or 63% CO/37% PES and TNT PES.

Another aspect of the present disclosure relates to a composition comprising coffee grounds for the treatment of wounds; in particular exposed wounds, more particularly for odour control; wherein the composition comprising coffee grounds is administered in a transdermal dressing comprising: an absorbent layer comprising an odour-inhibiting mixture; wherein the odour-inhibiting mixture comprises coffee grounds. In preferred embodiments, the composition may comprise the features described above for the dressing of the present embodiment.

Also described is the use of the dressing described in any of the previous embodiments as a wound odour absorber.

Also described is a method for producing a wound dressing in accordance with the present disclosure which comprises the following steps: preparing the odour-inhibiting mixture with coffee grounds; placing the odour-inhibiting mixture in the absorbent layer; applying the absorbent layer on a textile substrate; drying in the oven, preferably infrared (IR) at 100° C., the substrate with the composition; pressing or laminating the structure in order to obtain a dressing, preferably pressing at 110° C. with a pressure of 6 bar for 1 minute.

In an embodiment, the method for producing a dressing according to the previous embodiment in which the coffee grounds are ground at 400 rpm, for 30 minutes, whenever the inhibiting mixture is prepared and the textile is further functionalized.

In an embodiment, the preparation of the binding agent comprises the following steps: preparing a mixture of water or a phosphate buffer solution; adding chitosan and acetic acid under mechanical/magnetic stirring until the chitosan is well dissolved and the solution is homogeneous; adding the coffee grounds to the chitosan solution; well mixing using a mechanical stirrer; adding the silane and keeping stirring until the mixture is well homogeneous.

The present embodiments have several advantages, which are listed below.

### BRIEF DESCRIPTION OF THE FIGURES

For an easier understanding, figures are herein attached, which represent preferable embodiments which are not intended to limit the object of the present description.

FIG. 1—Illustration of an embodiment of the results of the reduction capacity of isovaleric acid (IVA) by the different layers of the commercial dressings Carboflex® and Actisorb®.

FIG. 2—Illustration of a preferred embodiment of the techniques used in the coating: (a) there is the film applicator, (b) the knife coating and (c) the Meyer bar is present.

FIG. 3—Illustration of an embodiment of an intersection edge of two fitted square sides of the half cube.

FIG. 4—Illustration of an embodiment of the results of the IVA reduction capacity by TNT PES functionalized with the various compounds analysed and the respective formulation of the present embodiment.

FIG. 5—Illustration of an embodiment of the results obtained from IVA reduction capacity by the 100% CO fabric functionalized with the various compounds analysed and the respective controls under study.

FIG. 6—Illustration of an embodiment of results obtained from IVA reduction capacity by the 100% CO and CO/PES fabric functionalized with different amounts of grounds and respective controls under study.

FIG. 7—Illustration of an embodiment of the results obtained from the IVA reduction capacity by the CO/PES fabric functionalized on one side and on both sides with the formulation of ground and unground coffee grounds and respective controls.

FIG. 8—Illustration of an embodiment of the IVA reduction capacity by the 100% CO fabric functionalized with the formulation of grounds at different concentrations of silane and respective controls.

FIG. 9—Illustration of an embodiment of the IVA reduction capacity by the CO/PES fabric functionalized with the formulation of grounds at different concentrations of silane and respective controls.

FIG. 10—Illustration of an embodiment of the IVA reduction capacity by the prototypes of dressings developed with the outside in TNT, inner layer 100% CO or CO/PES with formulation and the respective complete dressings.

FIG. 11—Illustration of an embodiment of the IVA reduction capacity by the prototypes of complete dressings developed with the outside in TNT and/or CO/PES and respective controls, on different days and by different operators.

FIG. 12—Illustration of an embodiment of the IVA reduction capacity over 24 hours, by the Actisorb® dressing and the two dressings developed.

FIG. 13—Illustration of an embodiment of the granulometries of ground coffee grounds.

FIG. 14—Illustration of an embodiment of the diameter of the wire used in the rod of the Meyer bar.

### DETAILED DESCRIPTION

The present disclosure relates to a wound treatment dressing, in particular for the absorption of odours in wounds, including pressure ulcers, leg ulcers, cancerous wounds, diabetic foot ulcers, burns, traumatic or surgical wounds, which comprises an odour-inhibiting mixture with coffee grounds.

The present disclosure describes a wound treatment dressing comprising: an absorbent layer comprising an odour-inhibiting mixture; wherein the odour-inhibiting mixture comprises coffee grounds. In an embodiment, the granulometry of ground coffee grounds varies between 3-5000 μm; preferably between 4-4000 μm; more preferably 5-3000 μm and the coffee grounds are dried and/or ground. The dressing further comprises an outer layer comprising a textile substrate, which serves as a support for the absorbent layer; an absorbent layer comprising a textile substrate, which serves as a support for the inhibiting mixture. The odour-inhibiting mixture comprises an adhesion agent, where the odour-inhibiting mixture comprises 0.5-5% (V/V_{inhibiting mixture}) of the adhesion agent; and 20-25% (W/V_{inhibiting mixture}) of coffee grounds. In an embodiment, the odour-inhibiting mixture further comprises a dispersant, a binding agent and a buffer solution.

The present disclosure describes a wound treatment dressing comprising: an absorbent layer comprising an odour-inhibiting mixture; wherein the odour-inhibiting mixture comprises coffee grounds.

In an embodiment, the wound treatment dressing of the present disclosure comprises a granulometry of ground coffee grounds that ranges from 5-5000 μm and are dried/ground.

In an embodiment, the dressing further comprises: an absorbent layer comprising a textile substrate, which serves as a support for the inhibiting mixture. It also describes a method for producing the dressing that comprises the following steps: preparing the odour-inhibiting mixture with coffee grounds; placing the odour-inhibiting mixture in the absorbent layer; applying the absorbent layer on a textile substrate; drying in the oven, preferably infrared at 100° C., the substrate with the composition; pressing or laminating the structure in order to obtain a dressing, preferably pressing at 110° C. with a pressure of 6 bar for 1 minute.

In order to compare the performance of the present disclosure’ dressings, two types of commercial dressings were analysed, both indicated for the treatment of exudates and the management of chronic wound odour. These commercial dressings feature a layer of activated carbon as an odour-absorbing substrate. One of the main objectives of this disclosure consisted in the replacement of activated carbon by coffee grounds, since these show a high absorption/neutralization area and, at the same time, allowing to add value to a by-product of the coffee industry.

In an embodiment, the commercial dressings analysed were Carboflex® and Actisorb Silver 220®, marketed by the international companies Convatec® and Systagenix®, respectively.

In an embodiment, in order to evaluate the odour retention capacity of the commercial dressings studied, the gas chromatography technique with flame ionization detection (Gas Chromatography—Flame Ionization Detector, GC-FID) was used according to a procedure adapted from ISO 17299-3:2014—Determination of deodorant properties—Part 3: Gas chromatography method. The gas chromatograph (GC) with flame ionization detector used corresponds to the 2010 Plus model of the Shimadzu brand. The capillary column is from the brand Teknokroma, model Meta.X5, nonpolar, 50 m long, with an internal diameter of 0.20 mm and a film thickness of 0.33 μm. The chromatograph has a “split/splitless” type injector, set to a split ratio of 1:5. The results were obtained using the GC Solution version 2.4 software, specific from Shimadzu.

Commercial dressings, despite being considered efficient in terms of absorbing chronic wound exudates, after 24 hours in contact with the wounds have the problem of releasing the unpleasant odour of the exudate into the surrounding environment. Therefore, the IVA retention capacity of the different layers of the commercial dressings under analysis was evaluated, as shown in FIG. 1. In the scope of this disclosure, only IVA was used, since it is one of the odorous compounds frequently found in the beds of patients with chronic wounds who, in turn, have bacterial colonization or infection.

In an embodiment, as shown in FIG. 1, it appears that commercial dressings show very similar results, in terms of the reduction of this odorous marker, by the different layers. Between layers it is also observed that there are no significant differences in the percentage of reduction in IVA, for both commercial dressings.

In an embodiment, taking into account that the minimum percentage of IVA reduction, referred to by the standard, for a fabric (dressing) to be considered deodorant regarding this odour, is 85%, it can be said that both dressings present this property. However, after each test, the textile layers were smelled and it was found that the outer layers of both dressings absorbed and retained the odour of the analysed odorous compound. This situation may mean that this layer acts as a barrier in the interaction of the compound with the inner layer (of activated carbon), promoting a lesser reduction of the odours released from the exudate of chronic wounds, when the dressing is complete. Regarding the inner layer, it was found that it had no smell after the test, which indicates that this layer of activated carbon has the capacity to neutralize the odour. The higher percentage of IVA reduction for the inner layer (≈98%), followed by the complete dressing (≈94%) and, in turn, the outer layer (≈90%) proves what has been stated above. In general, it is concluded that, in terms of the capacity to reduce odours, the two commercial dressings behave similarly.

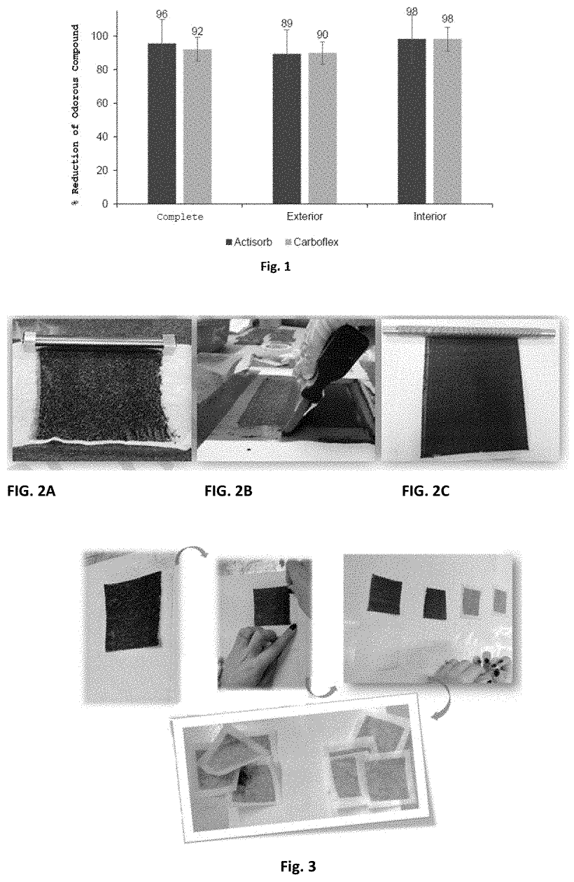

In an embodiment, for the incorporation of coffee grounds into the textile substrate, the use of the Meyer bar, with a thickness of 100 μm, was used. This technique made it possible to easily obtain a uniform coating with the desired thickness, as shown in FIG. 2. In this way, the Meyer bar was used in the incorporation of formulations with coffee grounds, in the different textile substrates. After the formulation was applied to the textile support, the coating was subjected to drying in an IR oven at 100° C. Finally, the samples were pressed at 110° C., with a pressure of 6 bar for 1 minute.

In an embodiment, the textile material with coffee grounds was developed using the textile substrates of 100% (V/V) CO, 63% (V/V) CO/37% (V/V) PES and TNT PES.

In an embodiment, several formulations were evaluated to define the most suitable one either in terms of spreading, or in terms of the amount of grounds and their effect on the capacity to reduce odours.

In an embodiment, the addition of thickeners such as cellosize (hydroxymethylcellulose) and CMC (carboxymethylcellulose) to the coffee grounds was assessed, however the amounts tested made it difficult to spread the formulation.

In an embodiment, the presence of a binder such as chitosan, glucomannan (konjac fiber) and impranil was also assessed. Glucomannan, although promoting a good texture to the formulation, acts as a prebiotic agent stimulating the growth and/or activity of certain bacteria. In this sense, it is not at all advisable in medical terms, as the formulation may be in contact with a wound. Impranil is an acrylic polymer resin that creates a film on the textile support, preventing to some extent the absorption of odours by the coffee grounds. Regarding chitosan, its addition makes the formulation with a viscosity suitable for spreading and still provides antimicrobial properties, important conditions considering the application purpose. However, the solution of chitosan in acetic acid with the coffee grounds is not sufficient for the formulation to have a good adhesion and uniformity on the textile substrate.

In an embodiment, taking into account these problems, the addition of a dispersant, such as TEGO WET 240 (highly active compound based on siloxane), and an adhesion agent, such as silane, was evaluated. The dispersant mentioned, despite improving the texture of the formulation with the coffee grounds and its spreading, is a copolymer of polyester siloxane. As such, it is a very reactive compound, so it has a high odour reduction capacity. In this sense, its presence masks the effect of the grounds, which is not intended with the present disclosure. Regarding silane, its presence has made the adhesion of grounds to the textile more efficient. Although at concentrations between 5-10% (V/V) it has the same effect as TEGO, in terms of the capacity to reduce odours, at low concentrations (0.1-3% (V/V)) this is not found, not influencing the effect of coffee grounds.

In an embodiment, in terms of the capacity to reduce odours, grinding or not grinding did not show significant differences. However, the uniformity of the dispersion of the formulation applied to the textile improves considerably when using ground grounds. Therefore, it was decided to grind the coffee grounds in a ball mill at 400 rpm, for 30 minutes, whenever the textile was functionalized.

In an embodiment, the odour-inhibiting mixture with better results comprises 20-25% w/Vₜₒₜₐₗ ground coffee grounds (SCG) in a 1% (w/V) solution of chitosan (Cs) in acetic acid (CH₃COOH 1% (V/V)) and 0.5% (V/V) silane. Due to the acidity of this formulation with a pH≈4, it was decided to bring the pH of the solution to 7, taking into account the possible contact with the skin and/or exudate from wounds. Thus, the amount of acetic acid (CH₃COOH) used was reduced to only 0.25% (V/V), using the phosphate buffer solution having pH≈7.4 in complementary manner.

In an embodiment, for the development of this embodiment, the inner layer of activated carbon of the commercial dressings was replaced by a coating with coffee grounds. In this sense, the present embodiment follows a structure similar to the dressings already existing on the market, being comprised by an inner layer and an outer layer.

In an embodiment, FIG. 3 shows how to make an embodiment of the dressings of the present embodiment, using a thermally adhesive tape for their closure.

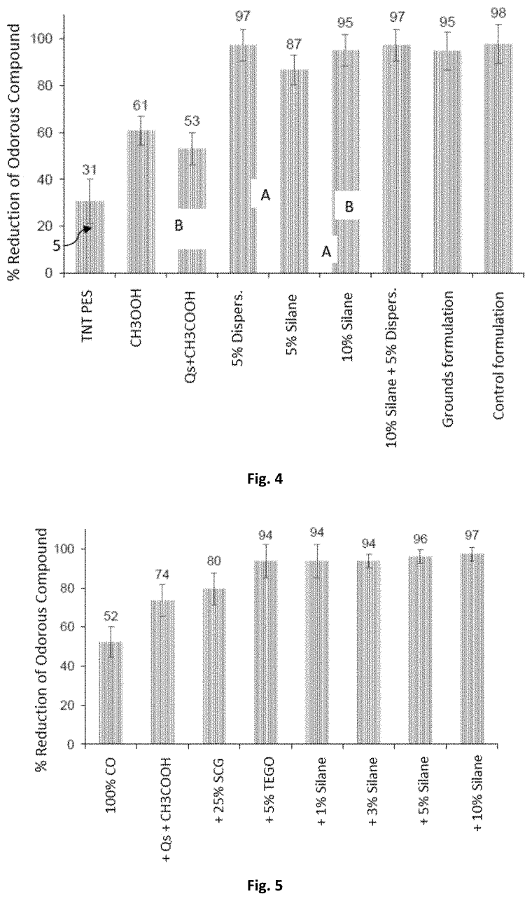

In an embodiment, through the analysis of FIG. 4, it is possible to verify that both chitosan and acetic acid promote a small increase in the capacity of intrinsic reduction of IVA by TNT PES, although this is not significant. In contrast, 5% (V/V) of TEGO (dispersant) impregnated in the fabric promotes a considerable increase, which indicates that this compound masks the effect of coffee grounds when added together. The same occurs with 5% (V/V) and 10% (V/V) silane, although 5% (V/V) silane shows a lesser effect, which in a smaller percentage may be useful for binding the grounds to the textile and not masking the reduction capacity of the grounds themselves. These two compounds together provide a high IVA reduction capacity, taking into account their individual behaviors. The reduction capacity of the control fabric (everything but the grounds) and the fabric with the formulation had already been analysed and, in this case, it is present only for comparison.

In an embodiment, the results in FIG. 5 suggest that the effect of both compounds, TEGO (dispersant) and silane, is the same on the different substrates analysed. It is further noted that when adding TEGO to the fabric with chitosan and grounds (SCG), the percentage of IVA reduction considerably increases. Thus, it is confirmed that TEGO masks the effect of grounds, as its addition immediately promotes a high IVA reduction capacity. Regarding silane, it was found that in the range of 1-10% (V/V), together with the other compounds, its IVA reduction effect is practically the same and quite high, which means that the formulation would already be saturated in terms of odour reduction capacity with the addition of TEGO (5% (V/V).

In an embodiment of the present embodiment, it was decided to apply a formulation with only coffee grounds in a 1% (w/V) solution of chitosan in acetic acid, in 100% (w/w) CO and 63% (w/w) CO/37% (w/w) PES fabrics. In order to compensate for the removal of dispersant and silane in the formulation, it was decided to increase the amount of coffee grounds to 30% w/Vₜₒₜₐₗ.

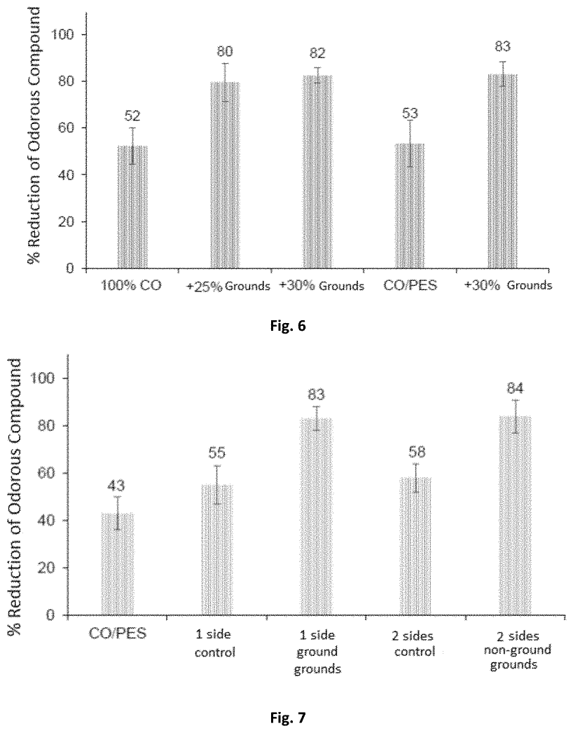

In an embodiment, the results of the chromatographic analysis, in terms of the odour reduction capacity of these samples and the respective controls without any type of coating, are shown in FIG. 6. As can be seen, for the substrate 100% (w/w) CO, there was no considerable increase when incorporating a greater amount of coffee grounds, so it was decided to continue with 25% w/Vₜₒₜₐₗ of coffee grounds.

In an embodiment, for the CO/PES blend fabric, a behavior similar to 100% (w/w) CO fabric is observed, as can be seen in FIG. 7. However, the intrinsic reduction capacity of the controls, both with one and with two sides of chitosan, is less than in relation to the 100% (w/w) CO fabric. This result is advantageous, since it makes the effect of grounds more noticeable.

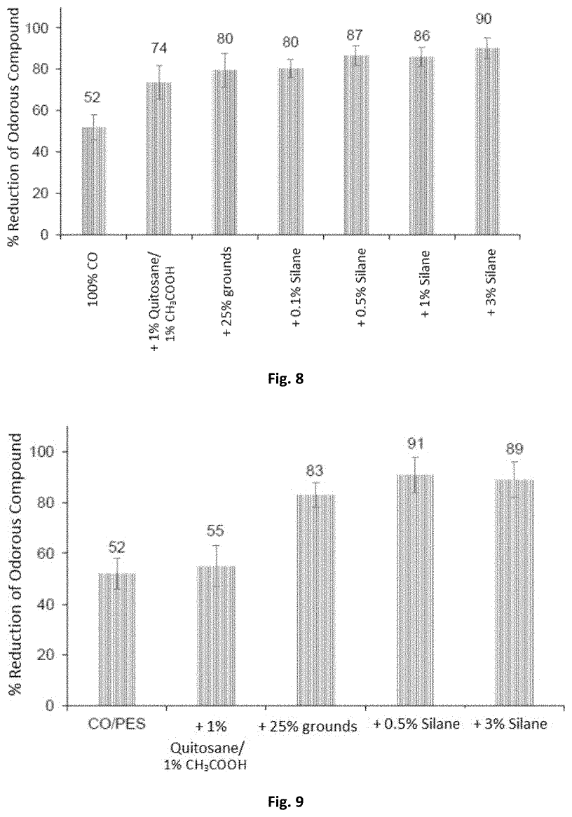

In an embodiment, as shown in FIG. 8 and FIG. 9, it appears that silane, in the concentrations tested and for both fabrics, does not have such a marked effect in terms of IVA reduction as in the concentrations previously tested. In addition to reaching a value close to that mentioned in the standard for the reduction of this marker, with these concentrations, an excellent formulation uniformity for spreading over the textile substrate is also obtained. Among the tested concentrations, and taking into account the values obtained for each one, it was decided to proceed with the use of 0.5% (V/V) of silane in future formulations. This is because, regarding the formulation of 0.1 and 1% (V/V) of silane, it reaches a value close to each other and ideal in relation to the standard criterion; and in comparison with the that of 3% it also reaches a close reduction value. In addition, the use of a lower concentration makes the formulation more beneficial for possible direct contact with the skin.

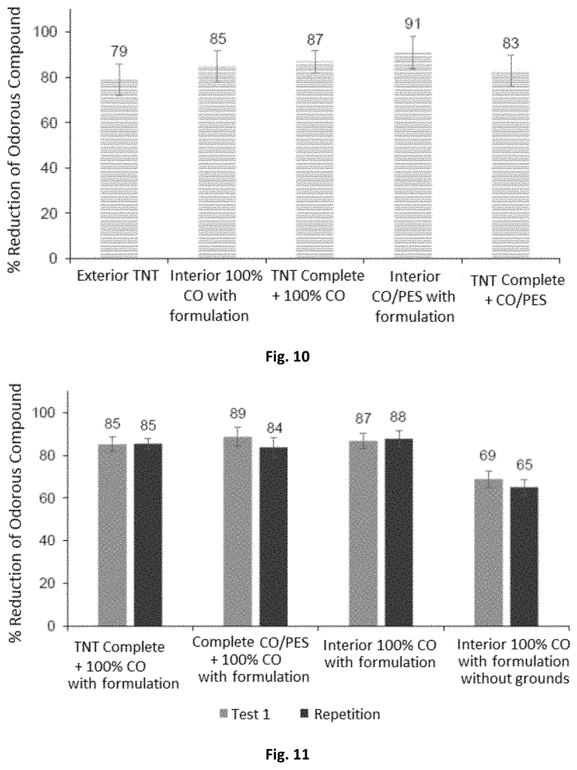

In an embodiment, FIG. 10 shows the results obtained for the dressing developed with the outer layer of TNT and the inner layer of 100% CO or CO/PES. It is possible to verify that, in dressings developed in comparison with the commercial ones, the outer layer has a IVA reduction capacity lower than the inner layer, allowing it to also act in the case of the complete dressing. Both TNT PES with 100% (w/w) CO as well as functionalized CO/PES show significant results in reducing this marker, taking into account the criterion defined in the standard, despite the latter being relatively impaired when complete.

In an embodiment, after each test, the wound dressings of the present embodiment were smelled and it was found that these neutralized/softened the odour of the odorous compound analysed. The same was not seen with the commercial dressings, so these prototypes showed the potential to overcome this problem.

In an embodiment, another way of making the dressings with the coffee grounds covered the application of them, loose, inside a kind of bag, which was later covered with an outer layer, using the same combination of fabrics. In the same study, it was also possible to evaluate the performance of this inner layer, with the loose grounds, individually, for direct application on chronic wounds. The amount of loose grounds added was 5 grams in little bags with approximately 50 cm² of area.

In an embodiment, after analysing various parameters in terms of the form and quantity of grounds, the components of the formulation, the fabrics to be used and the most appropriate functionalization technique, the remaining studies followed with only 2 of the developed dressing prototypes. Namely, dressings with the outer layer in CO/PES coupled with the inner layer 100% (w/w) CO with formulation and the outer layer TNT with the inner layer 100% (w/w) CO coated with the formulation under study. The dressing with the outer layer in 100% (w/w) CO and the inner layer of CO/PES coated with the formulation was discarded from the following tests, considering that in terms of medical application it would not be so appropriate.

In an embodiment, FIG. 11 shows that there is consistency in the results obtained, inasmuch as these tests were performed 3 months apart and by different operators. In addition, the preparation of the formulations and the incorporation into the textile substrate 100% CO was also carried out on different days. Therefore, it can be said that the method of incorporating coffee grounds into the textile, the preparation of the dressings and their capacity in terms of the percentage of odour reduction is stable and controlled, not varying significantly.

Another problem of the art was that the commercial dressings, after some time of use, didn't have the capacity to neutralize the bad odour released by exudates of chronic wounds. In this sense, it was decided to evaluate this parameter using two different tests.

In an embodiment, the first trial consisted of assessing the odour retention capacity of the dressings (Actisorb® and the other 2 developed), over a period of 24 hours, allowing to monitor their effectiveness. To this end, 85 μl of IVA were injected in an Erlenmeyer flask, in each dressing, simulating the amount of exudate a leg pressure ulcer releases every half hour [L. F. B. Giraldo, “Extraction and characterization of polysaccharides and phenolic compounds from spent coffee grounds and their incorporation into edible films/coatings for food applications”, 2016]. The values were obtained after two hours, consecutive with each injection, until reaching 24 hours of contact with the dressings.

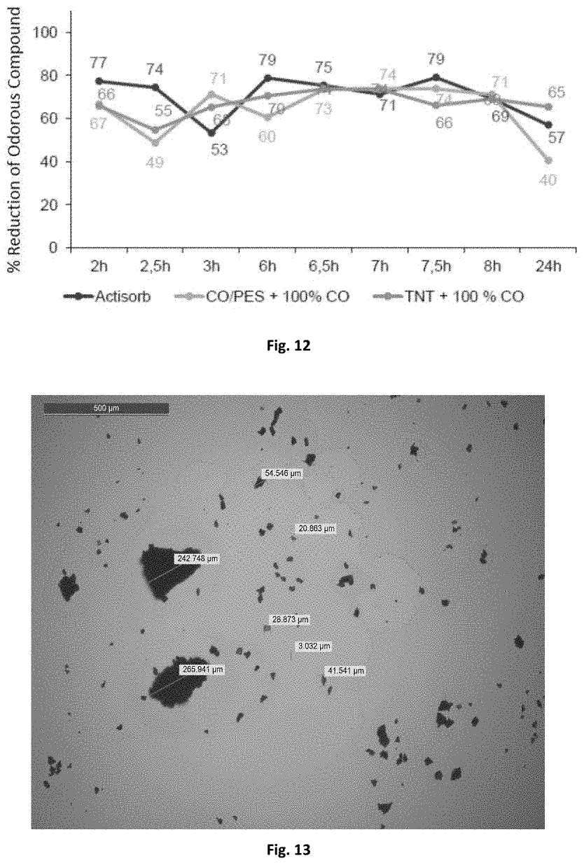

In an embodiment, the results shown in FIG. 12 show that the developed dressings exhibit a behaviour similar to the commercial dressing, in terms of odour reduction, during the 24 h of simulation. It should be noted that the dressing exterior in TNT and with the interior of 100% (w/w) CO with formulation has a more linear trend, over time and that after 24 hours the reduction capacity exceeds that of the commercial dressing.

In an embodiment, the odour of the dressings was qualitatively assessed throughout the test—characterized as a weak, medium, or strong odour, in order to determine their neutralization capacity (Table 1). In summary, it was noticed that the exterior dressing in TNT and with the interior of 100% CO with formulation only started to present a strong odour of IVA after 24 h, while the other two already released a strong odour to the surrounding air after 7 h.

TABLE 1
Qualifying evaluation of the IVA odour in the 24 h test, both for
the commercial dressing and for the two developed prototypes.

| Dressings | Qualification of IVA odour |
|---|---|
| Actisorb ® | Strong odour from 7 h |
| Exterior TNT + Interior 100% CO with | Strong odour after 24 h |
| formulation | |
| Exterior CO/PES + Interior 100% CO with | Strong odour from 7 h |
| formulation | |

From the obtained results, it appears that the developed prototypes have an absorption capacity similar to the Actisorb® commercial dressing, despite being slightly lower. Regarding the Carboflex® commercial dressing, the absorption capacity of the developed dressings is much lower. This result can be explained by the fact that this dressing consists of 4 layers, allowing to absorb a greater amount of solution, compared to the developed prototypes. Anyway, as the developed dressings are structurally similar to Actisorb®, it can be said that in terms of absorption capacity the developed prototypes show satisfactory results. In general terms, dressings with greater absorption capacity are those that have the outside in TNT and an inner layer with grounds in formulation or loose.

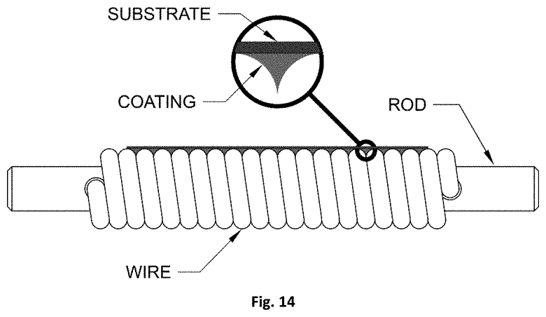

In the present embodiment, FIG. 14 shows a Meyer bar as a technology for applying coatings, similar to a film applicator.

In this coating process, the formulation is deposited on the substrate and is displaced by means of a dosing rod wrapped in wire (Meyer bar). The Meyer bar allows the desired amount of coating to remain on the substrate and the amount is determined by the diameter of the wire used in the rod, in this case a wire with a diameter of 100 μm was used.

In an embodiment, the wound treatment dressings described in the present disclosure were sterilized by gamma radiation in the Unidade Tecnológica de Radioesterilização of the Instituto Superior Técnico with different doses of radiation (5, 15 and 25 kGy). The evaluation of the microbial load before and after sterilization was carried out at the Centro Tecnológico CITEVE—Tecnologia Têxtil (reports attached).

TABLE 2
Results of the microbiological control of the dressings of the
present disclosure by the Portuguese Pharmacopoeia 2.6.12

| | | Total No. of | | |
|---|---|---|---|---|
| | | viable aerobic | Bacteria, | Fungi, |
| | | germs (CFU/g) | (CFU/g) | (CFU/g) |
| 1^{st} test | Without | 2166 | 1770 | 396 |
| | sterilization | | | |
| 2^{nd} test | Without | 1632 | 452 | 1180 |
| | sterilization | | | |
| | 5 KGy | 611 | 608 | 3.33 |
| | 15 KGy | 85 | 73.4 | 11.3 |
| | 25 KGy | 16 | 8.95 | 6.69 |

The term “comprises” or “comprising” when used herein is intended to indicate the presence of stated features, elements, integers, steps, and components, but not to preclude the presence or addition of one or more other features, elements, integers, steps and components, or groups thereof.

The embodiments described are combinable with each other. The present invention is not, of course, in any way restricted to the embodiments described in this document and a person with average knowledge in the art will be able to foresee many possibilities for modifying it and replacing technical features with equivalent ones, depending on the requirements of each situation, as defined in the appended claims.

The following claims define further embodiments of the present description.

1. A wound treatment dressing comprising:
an absorbent layer comprising an odour-inhibiting mixture,
wherein the odour-inhibiting mixture comprises coffee grounds,
wherein the granulometry of the ground coffee grounds varies between 3-5000 μm,
wherein the odour-inhibiting mixture comprises 10-40% (W/V_{inhibiting mixture}) of coffee grounds, and
wherein the odour-inhibiting mixture comprises an adhesion agent, a dispersant, a binding agent or a buffer solution, or mixtures thereof.

2. The dressing according to claim 1, wherein the granulometry of the ground coffee grounds varies between 4-4000 μm.

3. The dressing according to claim 1, wherein the odour-inhibiting mixture comprises 15-30% (w/V_{inhibiting mixture}) of coffee grounds.

4. The dressing according to claim 1, wherein the absorbent layer comprises between 0.05 to 5 g of coffee grounds per cm² of absorbent layer.

5. The dressing according to claim 1, wherein the coffee grounds are dried and/or ground.

6. The dressing according to claim 1, wherein the dressing further comprises an outer layer comprising a textile substrate, which serves as a support for the absorbent layer carrying the inhibiting mixture.

7. The dressing according to claim 1, wherein the absorbent layer further comprises superabsorbent polymers.

8. (canceled)

9. The dressing according to claim 1, wherein the dressing comprises 0.5-5% (V/V_{inhibiting mixture}) of bonding agent.

10. The dressing according to claim 1, wherein the adhesion agent is selected from a list consisting of: a highly active matrix based on anionic, cationic, non-ionic, amphoteric agents, and combinations thereof.

11. The dressing according to claim 1, wherein the binding agent is selected from a list consisting of: chitosan, phosphate buffer solution, gelatin, pectin, cellulose, cellulose derivatives, glucomannan, acrylic emulsions, styrene-butadiene emulsions, styrene-acrylic emulsions, polyurethane-based dispersions, and combinations thereof.

12. The dressing according to claim 1, wherein the dispersant is selected from a list consisting of: polyester, siloxane, polyphosphates, polyacrylates, and combinations thereof.

13. The dressing according to claim 1, wherein the absorbent layer further includes an active ingredient, a moisturizer, a disinfectant, an anesthetic agent or combinations thereof.

14. The dressing according to claim 1, wherein the absorbent layer is coated through a Meyer bar and/or film applicator.

15. (canceled)

16. The dressing according to claim 1, wherein the textile substrate is a natural fiber, a synthetic fiber or combinations thereof.

17. The dressing according to claim 1, wherein the pH range of the buffer solution is between 6 and 10.

18. The dressing according to claim 1, wherein the textile substrate comprises cotton, polyester, non-woven fabric, or combinations thereof.

19. A method for absorbing the odour of a wound in a subject, comprising administering to the subject a transdermal dressing wherein the transdermal dressing comprises:
an absorbent layer comprising an odour-inhibiting mixture;
wherein the odour-inhibiting mixture comprises coffee grounds,
wherein the granulometry of the ground coffee grounds varies between 3-5000 μm,
wherein the odour-inhibiting mixture comprises 10-40% (w/V_{inhibiting mixture}) of coffee grounds, and
wherein the odour-inhibiting mixture comprises an adhesion agent, a dispersant, a binding agent or a buffer solution, or mixtures thereof.

20. (canceled)

21. A method for producing a dressing according to claim 1, comprising the following steps:
preparing the odour-inhibiting mixture with coffee grounds;
placing the odour-inhibiting mixture in the absorbent layer;
applying the absorbent layer on a textile substrate;
drying in the oven, preferably infrared (IR) at 100° C., the substrate with the composition; and
pressing or laminating the structure in order to obtain the dressing, preferably pressing at 110° C. with a pressure of 6 bar for 1 minute.

22. The method for producing the dressing according to claim 21, wherein the coffee grounds are ground at 400 rpm for 30 minutes;
whenever the inhibiting mixture is prepared and the textile is further functionalized.

23. The method for producing the dressing according to claim 21, wherein the odour-inhibiting mixture comprises a binding agent and wherein preparation of the binding agent comprises the following steps:
preparing a mixture of water or a phosphate buffer solution;
adding chitosan and acetic acid under mechanical/magnetic stirring until the chitosan is well dissolved and the solution is homogeneous;
adding the coffee grounds to the chitosan solution;
mixing well using a mechanical stirrer; and
adding the silane and keeping stirring until the mixture is well homogeneous.
